(19) United States
(12) Patent Application Publication
(10) Pub. No.: US 2006/0084917 A1
(43) Pub. Date: Apr. 20, 2006

(54) Retractable syringe

(76) Inventor: Vincent Chen, Sanchung City (TW)

Correspondence Address: LEONG C LEI, PMB # 1008, 1867 YGNACIO VALLEY ROAD, WALNUT CREEK, CA, 94598, US

(21) Appl. No.: 10/892,101

(22) Filed: Jul. 16, 2004

(51) Int. Cl. A61M 5/00; A61M 5/32; A61M 5/31

(52) U.S. Cl. 604/110; 604/192; 604/243

(57) ABSTRACT

A retractable syringe is disclosed. The syringe includes a cap, a hub cover, a hub bottom, a gasket, a plunger and a barrel. Medicine for injection is placed within the barrel to proceed with an injection. After the injection is carried out by pulling back the plunger, an engaging button linked to the hub cover and hub bottom is pulled down and the needle mounted at the hub cover is hidden within the barrel. Then, the plunger is broken to avoid the needle from being accidentally pushed outward and to avoid recycling of the needle and the barrel.

### BACKGROUND OF THE INVENTION

(a) Technical Field of the Invention

This invention relates to a retractable syringe, in particular to one which, after being used once, will enable the needle to retract into the barrel whereupon the plunger will be broken to prevent the needle and the barrel from being re-used.

(b) Description of the Prior Art

According to a World Health Organisation report, about 8 to 16 million of the world's population have contracted Hepatitis B, and about 2 to 4.5 million have contracted Hepatitis C, and about 70,000 to 150,000 people have contracted AIDS as a result of using dirty needles.

A retractable syringe has the characteristic that the needle can be retracted into the barrel of the syringe to avoid oneself and other people from being accidentally hurt. Thus, the connection between the needle seat of the needle and the barrel has to have a pull resistance F, pressure resistance E, and reverse pull G, as shown in FIG. 2.

If (a) pull resistance F (3 kg/cm) is large, there is a risk of dislocation of the needle onto the patient after it has been used. If (b) pressure resistance E (3 kg/cm) is large, the needle may be slid into the barrel before it is injected into the patient. If (c) reverse pull G is small, the needle can be pulled back to the barrel. Accordingly, it may not be possible to have a syringe with large pressure resistance E and a small reverse pull G

FIG. 3 is a conventional back-pull type syringe having a needle seat A and a barrel B. Protrusion C and recess D are used for locking the needle seat and the barrel and the air tight seal of the barrel. This method solves the pull resistance F and pressure resistance E of the barrel, but the reverse pull G cannot be easily controlled. If there is a greater resistance between the needle seat and the barrel, the force needed for the needle seat to be pulled back to the barrel is greater. The larger the contact area, the larger the force exerted. This will be inconvenient for the user. If the resistance between the needle seat and the barrel is small, the reverse pull G is reduced but the air tight seal is poor. This may cause the needle to fall out onto the patient.

Accordingly, it is an object of the present invention to provide a retractable type syringe which can mitigates the above drawback.

### SUMMARY OF THE INVENTION

It is an object of the present invention to provide a retractable syringe comprising a cap having a cylindrical shape with a circular rim seat at one end and the external surface of the cap being formed with anti-slip elongated slots and the interior of the cap having a stepped circular hole and a semi-circular protruded rim. The retractable syringe further includes a hub cover having a stepped cylindrical post and a hole at the center thereof A reinforcing rib is provided to the stepped cylindrical post and an arch-shaped circular bottom seat is provided to the lower end of the stepped cylindrical post. At the half position of the circular bottom seat is formed and the lower section of the bottom seat has a recessed rim which is firmly engaged with the protruded rim being formed. The lower section of the circular bottom seat has an arch-shaped protruded rim for firm engagement with the recessed rim at the hub bottom

The inner circle of the circular bottom seat is provided with a guiding post and the lower section of the guiding post is formed as a protruded rim. The hub bottom extends the inwardly sloping bottom seat of the hub cover facilitating the engagement of the arch-shaped recessed rim and the protruded rim of the barrel. A cylindrical guiding holder is provided to the hub bottom which is mounted with the guiding post. A protruded rim is provided to the upper section of the inner circular of the guiding hole to control the distance of disengagement of the arch-shape recessed rim of the hub cover and the arch-shape protruded rim, and to fasten on the protruded rim of the hub cover facilitating the pulling of the hub cover back to the barrel. The inner section of the bottom seat has a conical hole and the upper section of the bottom seat is a recess which is in combination with the protruded rim of the hub cover and extends through the inwardly sloping bottom seat of the hub cover. The conical hole at the bottom seat is connected to a tapered circular hole. A barrel has a stepped cylindrical body having a hollow interior. The upper end of the inner opening is provided with a protruded rim for engagement with the recessed rim of the hub cover. The lower section of the inner hole is a protruded rim for engagement with the circular rim seat of the plunger to prevent the needle seat from being pushed outside of the barrel.

The oval shaped handle of the syringe is an elongated protruded strap for preventing slipping. The plunger has a conical engaging hook and the center of the plunger has a V-shaped slot and the conical engaging hook for engagement with the inner engaging hook of the hub bottom so as to pull the hub cover and the hub bottom back to the barrel. The outside of the circular thin rim seat is engaged with the protruded rim of the barrel to prevent the needle seat from being pushed out of the barrel. The bottom of the thin circular seat is a wing flap having a sectioned area for breakage, and at the joining of one wing flap with another wing flap, a circular post is provided for breaking. One end of the plunger has a circular handle with elongated protruded strap for prevention of slipping and a gasket having a cylindrical shape with an interior having a center hole for connection with the circular rim seat of the plunger. The circular protruded rim is used for water stopping.

The foregoing object and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective exploded view of the syringe in accordance with the present invention.

FIG. 1A is a perspective view of the syringe in accordance with the present invention.

FIG. 2 is a schematic view showing the application of the syringe in accordance with the present invention.

FIG. 3 is a sectional view showing the conventional syringe.

FIG. 4A is a perspective view of the cap of the syringe in accordance with the present invention.

FIG. 4B is another perspective view of the cap of the syringe in accordance with the present invention.

FIG. 4C is a front view of the cap of the syringe in accordance with the present invention.

FIG. 4D is a sectional view of the cap of the syringe in accordance with the present invention.

FIG. 4E is an enlarged view of the bottom portion of the cap of the syringe in accordance with the present invention.

FIG. 4F shows a top view of the cap of the syringe in accordance with the present invention.

FIG. 5A is a perspective view of the hub cover of the syringe in accordance with the present invention.

FIG. 5B is a reverse perspective view of the hub cover of the syringe in accordance with the present invention.

FIG. 5C is a front view of the hub cover of the syringe in accordance with the present invention.

FIG. 5D is a sectional view of the hub cover of the syringe in accordance with the present invention.

FIG. 5E is a top view of the hub cover of the syringe in accordance with the present invention.

FIG. 6A is a perspective view of the hub bottom of the syringe in accordance with the present invention.

FIG. 6B is a reverse perspective view of the hub bottom of the syringe in accordance with the present invention.

FIG. 6C is a front view of the hub bottom of the syringe in accordance with the present invention.

FIG. 6D is a sectional view of the hub bottom of the syringe in accordance with the present invention.

FIG. 6E is a top view of the hub bottom of the syringe in accordance with the present invention.

FIG. 7A is a perspective view of the barrel of the syringe in accordance with the present invention.

FIG. 7B is a reverse perspective view of the barrel of the syringe in accordance with the present invention.

FIG. 7C is a front view of the barrel of the syringe in accordance with the present invention.

FIG. 7D is a sectional view of the barrel of the barrel of the syringe in accordance with the present invention.

FIG. 7E is an enlarged view of the top portion of the barrel of the syringe in accordance with the present invention.

FIG. 7F is a top view of the barrel of the syringe in accordance with the present invention.

FIG. 8A is a perspective view of the plunger of the syringe in accordance with the present invention.

FIG. 8B is a reverse perspective view of the plunger of the syringe in accordance with the present invention.

FIG. 8C is a front view of the plunger of the syringe in accordance with the present invention.

FIG. 8D is a top view of the plunger of the syringe in accordance with the present invention.

FIG. 9A is a perspective view of the gasket of the syringe in accordance with the present invention.

FIG. 9B is another perspective view of the gasket of the syringe in accordance with the present invention.

FIG. 9C is a front view of the gasket of the syringe in accordance with the present invention.

FIG. 9D is a sectional view of the gasket of the syringe in accordance with the present invention.

FIG. 9E is a top view of the gasket of the syringe in accordance with the present invention.

FIG. 10 shows the injection step (1) in accordance with the present invention.

FIGS. 11A, 11B and 11C are sectional views showing the injection step (2) in accordance with the present invention.

FIGS. 11D, 11E and 11E are perspective views showing the injection step (2) in accordance with the present invention.

FIGS. 12A, and 12B are sectional views showing the injection step (3) in accordance with the present invention.

FIG. 12C is an enlarged view of the bottom portion of FIG. 12A in accordance with the present invention.

FIGS. 12D and 12E are perspective views showing the injection step (3) in accordance with the present invention.

FIG. 13 is a sectional view showing the combination of the hub cover and the hub bottom in accordance with the present invention.

### DETAILED DESCRIPTION OF THE PREFERRED EMBODIMENTS

The following descriptions are of exemplary embodiments only, and are not intended to limit the scope, applicability or configuration of the invention in any way Rather, the following description provides a convenient illustration for implementing exemplary embodiments of the invention. Various changes to the described embodiments may be made in the function and arrangement of the elements described without departing from the scope of the invention as set forth in the appended claims.

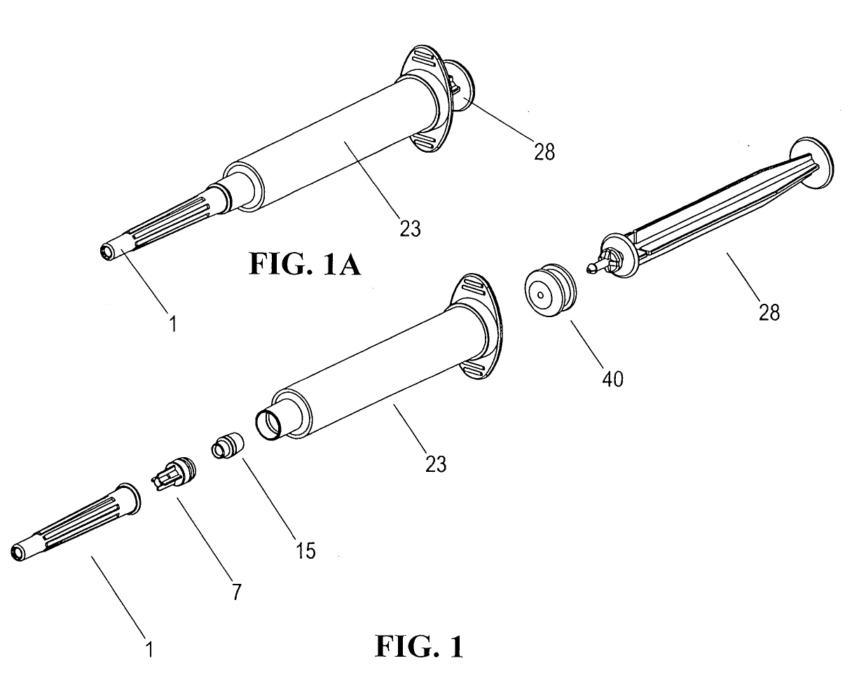

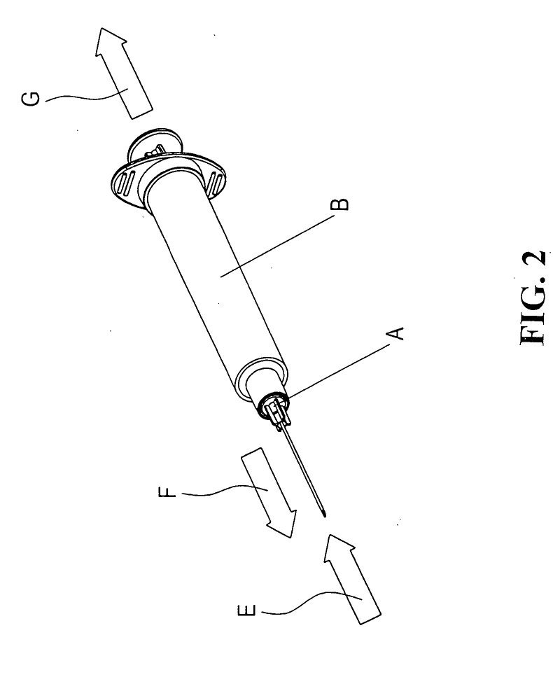

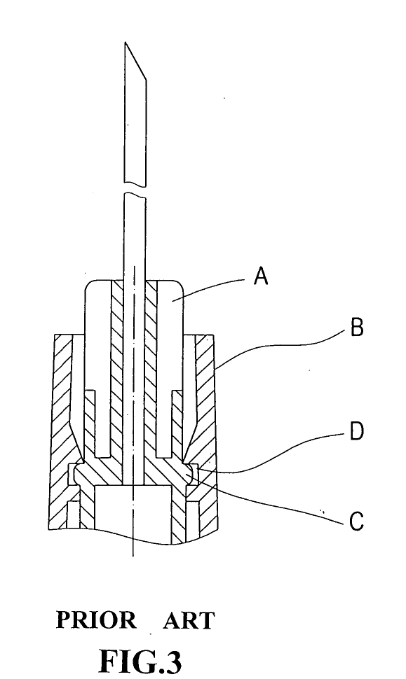

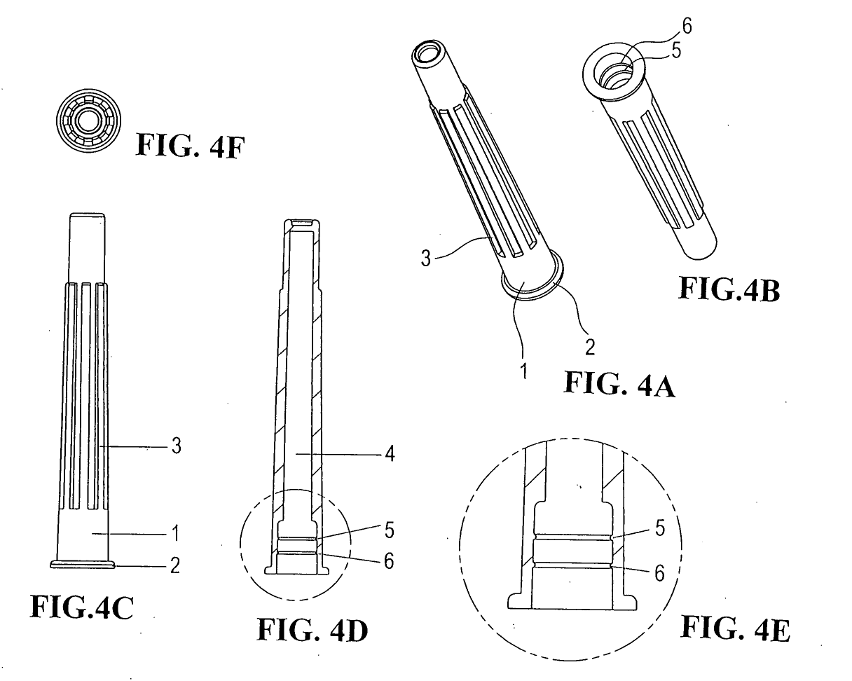

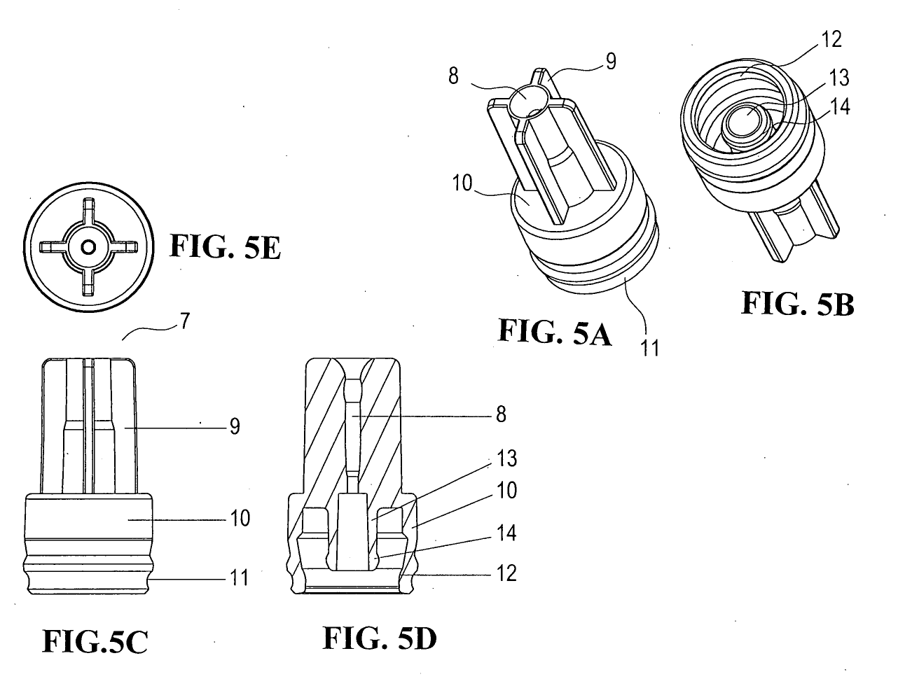

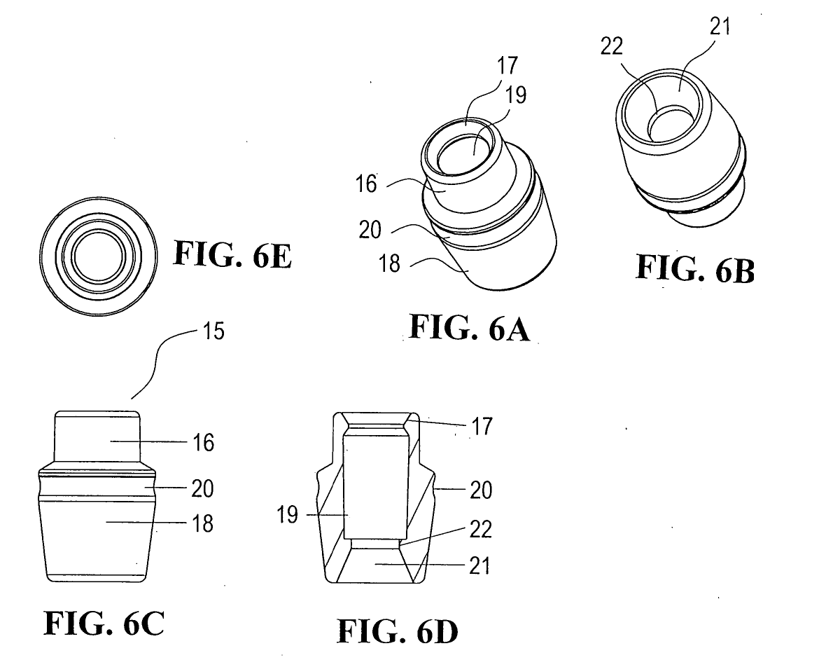

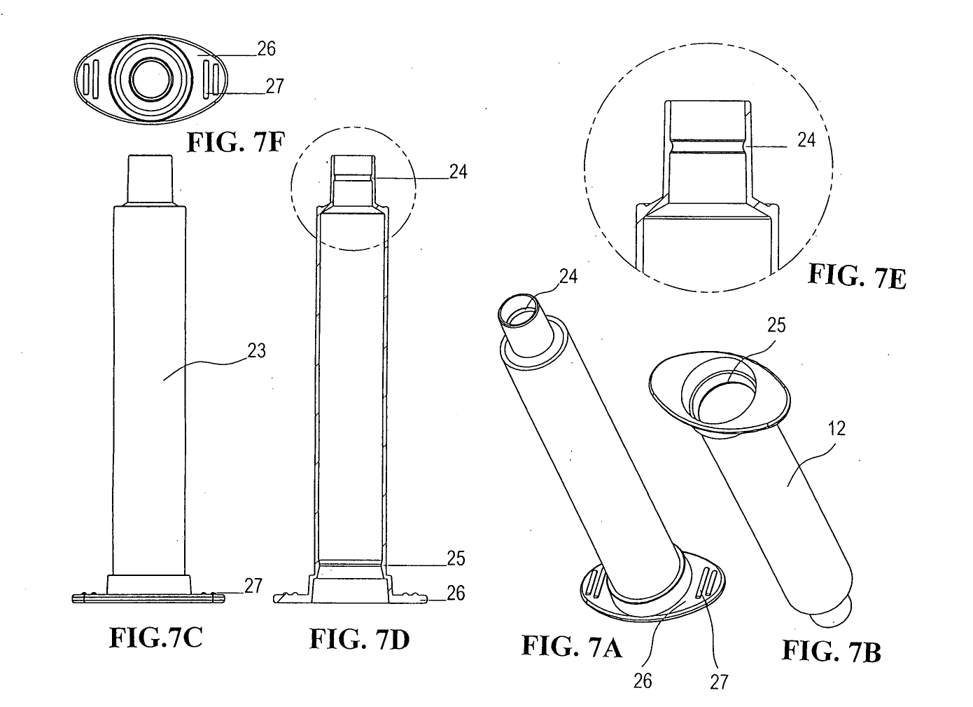

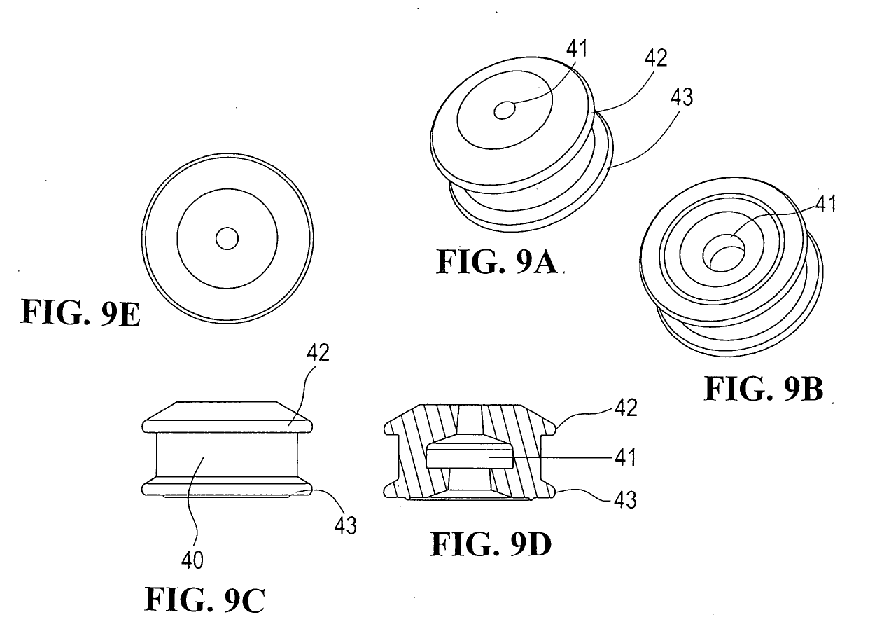

Referring to FIGS. 1, 1A, 4A, 4B, 4C, 4D, 4E, 4F, 5A, 5B, 5C, 5D, 5E, 6A, 6B, 6C, 6D, 6E, 7A, 7B, 7C, 7D, 7E, 7F, 8A, 8B, 8C, 8D, 9A, 9B, 9C, 9D, 9E, there is shown a retractable syringe comprising a cap 1 (shown in FIGS. 4A, 4B, 4C, 4D and 4E) having a cylindrical shape with a circular rim seat 2 at one end and the external surface of the cap being formed with anti-slip elongated slots 3 and the interior of the cap 1 having a stepped circular hole 4 and semi-circular protruded rim 5 and 6 for securing the needle seat; a hub cover 7 (FIGS. 5A, 5B, 5C and 5D) having a stepped cylindrical post and a hole 8 for placing needle and needle adhesive at the center thereof, a reinforcing rib 9 provided to the stepped cylindrical post and an arch-shaped circular bottom seat 10 being provided to the lower end of the stepped cylindrical post and at the half position of the circular bottom seat inwardly sloping being formed and the lower section of the bottom seat a recessed rim 11 which is firmly engaged with the protruded rim 24 (FIGS. 7, 7A, 7B, 7C, 7D, 7E and 7F) being formed, and the lower section of the circular bottom seat 10 having an arch-shaped protruded rim 12 for firm engagement with the recessed rim 20 (FIGS. 6A, 6B, 6C, 6D and 6E) at the hub cover 7, and the inner circle of the circular bottom seat 10 a guiding post 13 being provided and the lower section of the guiding post 13 being a protruded rim 14; the hub bottom 15 extending the inwardly sloping bottom seat of the hub cover 7 facilitating the engagement of the arch-shaped recessed rim 11 (FIGS. 5A, 5B, 5C and 5D) and the protruded rim 24 (FIGS. 7A, 7B, 7C, 7D, 7E and 7F) of the barrel 23 and a cylindrical guiding holder 16 being provided to the hub cover 7 which is mounted with the guiding post 1, a protruded rim 24 (FIGS. 7A, 7B, 7C, 7D, 7E and 7F) being provided to the upper section of the inner circular of the guiding hole to control the distance of disengagement of the arch-shape recessed rim 11 of the hub cover 7 and the arch-shape protruded rim 24 (FIGS. 7A, 7B, 7C, 7D, 7E and 7F), and to fasten with the protruded rim 24 (FIGS. 7A, 7B, 7C, 7D, 7E and 7F) of the hub cover 7 facilitating the pulling of the hub cover 7, back to the barrel, the inner section of the hub bottom 15, having a conical hole 19 and the upper section of the bottom seat being a recess which is in combination with the protruded rim 12 (FIGS. SA, 5B, 5C, 5D, 5E) of the hub cover 7 and extends the inwardly sloping bottom seat of the hub cover 7, the conical hole 19 at the bottom seat connected to a tapered circular hole 21.

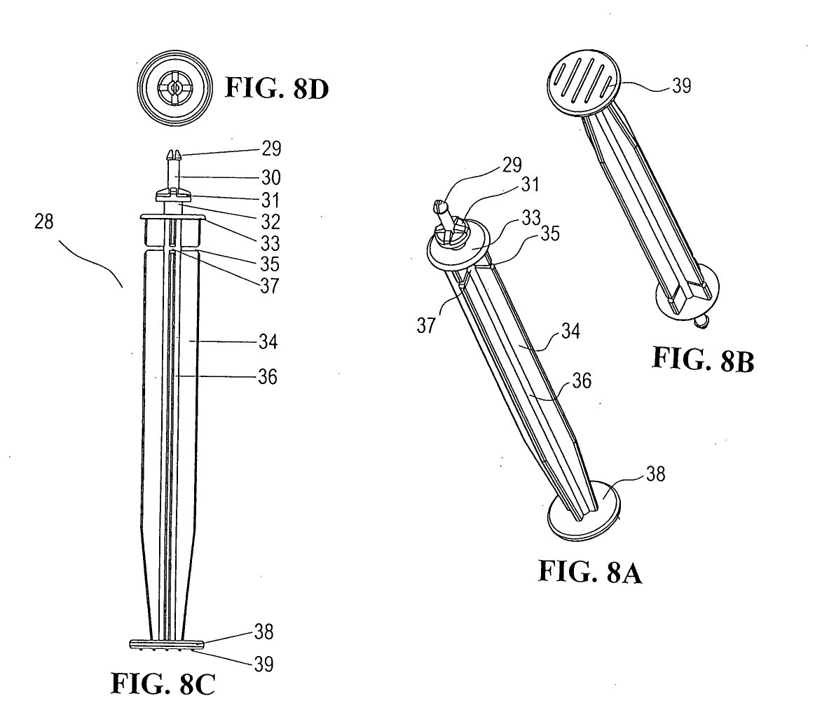

The barrel 23 has a stepped cylindrical body (FIGS. 7A, 7B, 7C, 7D, 7E and 7F) having a hollow interior. The upper end of the inner opening is provided with a protruded rim 24 for engagement with the recessed rim 11 of the hub cover and the lower section of the inner hole being a protruded rim 7 for engagement with the circular rim seat 33 (FIGS. 8A, 8B, 8C, 8D) of the plunger 28 to avoid the needle seat from being pushed outside of the barrel 23 and the oval shaped handle 26 of the syringe being elongated protruded strap 27 for preventing slipping.

The plunger 28 has a conic engaging hook 29 and the center of the plunger 28 is a V-shaped slot. The conic engaging hook 29 is for engagement with the inner engaging hook 22 (FIGS. 6A, 6B, 6C, 6D, 6E) of the hub bottom 15 so as to pull the hub cover 7 and the hub bottom 15 back to the barrel 23. The exterior of the circular thin rim seat 31 is engagement with the protruded rim 25 of the barrel 23 to prevent needle seat is pushed out of the barrel 23, and the bottom of the thin circular seat is wing flap 34 having a sectioned area for breakage, at the joining of one wing flap 34 with another wing flap 34. A circular post 36 is provided as breaking point 37.

One end of the plunger has a circular handle 38 with an elongated protruded strap 39 for preventing slipping. A gasket 40 has a cylindrical shape with an interior having a center hole for connection with the circular rim seat 31 of the plunger 28, and the circular protruded rim 43 is used for water stopping.

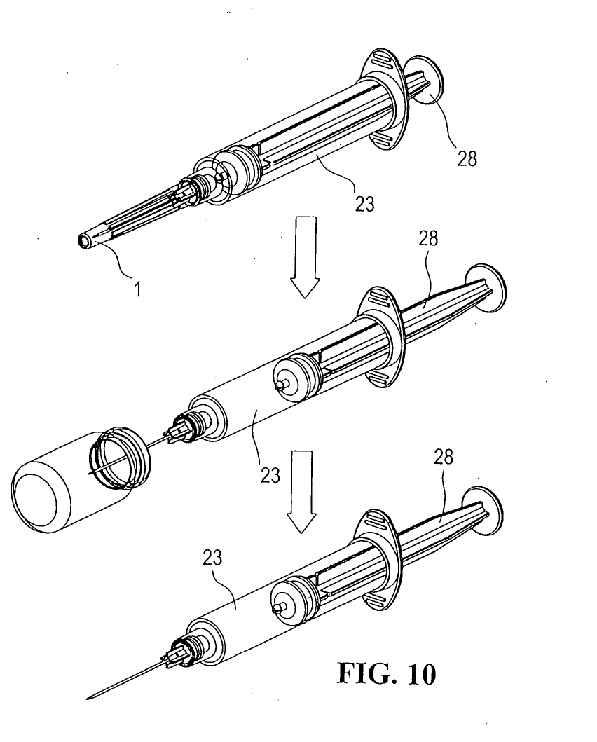

Referring to FIG. 10, this shows step 1 of the application of the syringe of the present invention, wherein the cap 1 is removed and medicine for injection is delivered into the barrel 23. After the medicine has been fully delivered, the syringe is ready for injection.

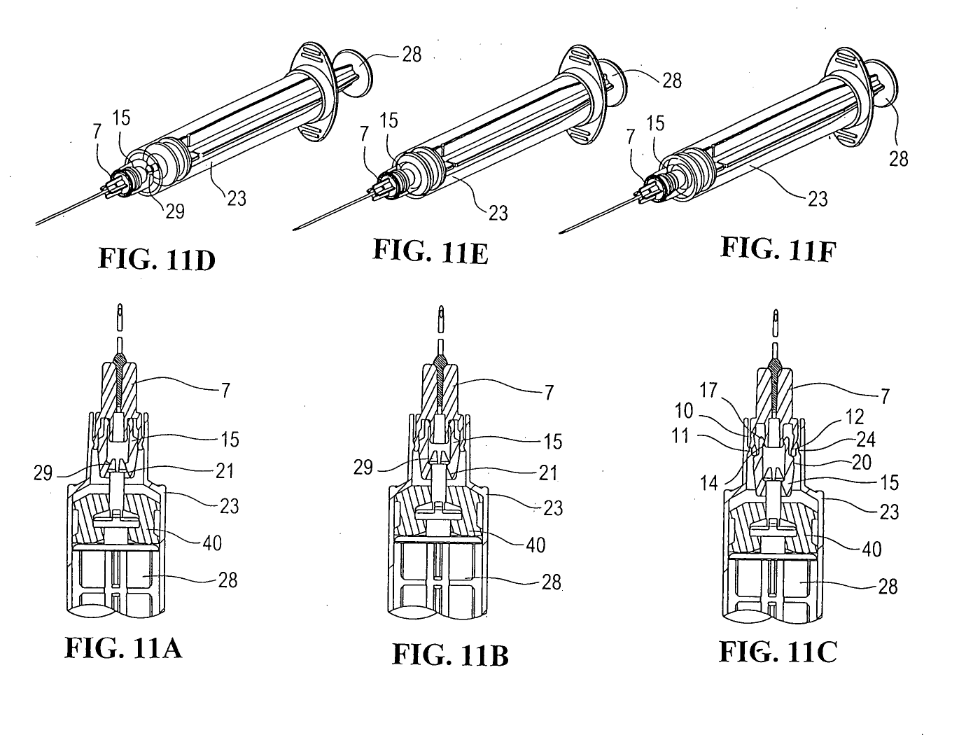

FIG. 11 shows step II application of the syringe. When injection of the medicine has been completed, the conical engaging hook 29 (FIGS. 8A, 8B, 8C, 8D) is in contact with the tapered circular hole 21 (FIGS. 6A, 6B, 6C, 6D, 6E). The small end hole of the circular hole 21 engages with the engaging hook 29. There is a resistance when the plunger 28 is pushed forward and it cannot enter the bottom needle seat 15.

Due to the V-shaped slot, when the plunger 28 moves forward, the engaging hook will retract inward and is engaged with the hub bottom 15. When the engaging hook is in engagement, the engaging hook will restore to its original size. At this instance, the plunger 28 can be subjected to back pull movement.

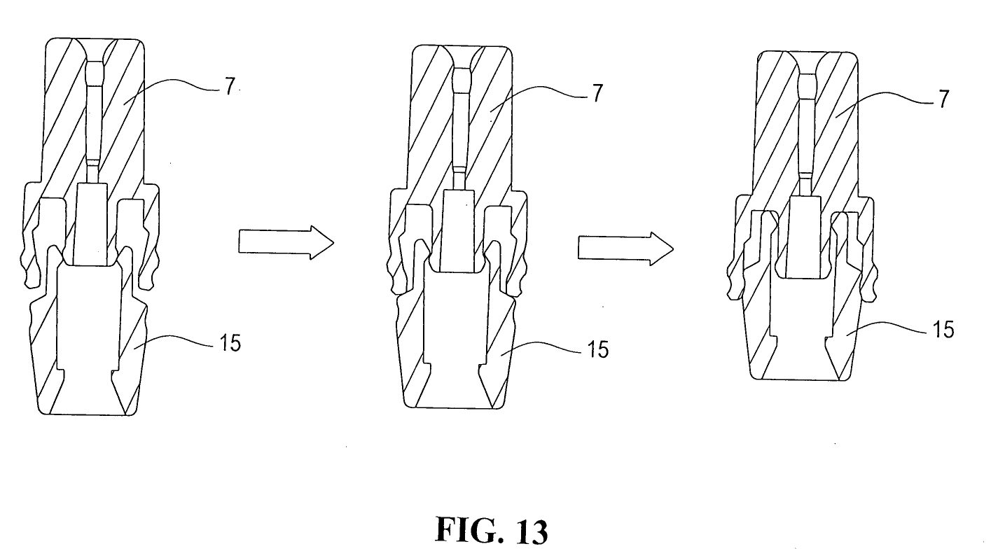

Due to the inward sloping of the circular bottom seat (FIGS. 5A, 5B, 5C, 5D, 5E) of the hub cover, the outward extension is generated by the bottom needle seat 15 (FIG. 13). There is a little resistance to the recessed rim 20 (FIGS. 6A, 6B, 6C, 6D, 6E) of the protruded rim 12 (FIG. 5A, 5B, 5C, and 5D). When the plunger is retracted a little, the hub bottom 15 will be dislocated from the hub cover 7. When the protruded rim 17 engages with the protruded rim 14, the hub bottom 15 is dislocated fully with the hub cover 7. At the same time, the bottom seat 10 of the hub cover 7 loses the stress produced by the hub bottom 15, and the inward sloping shape is restored. The resistance between the protruded rim 24 (FIGS. 7A, 7B, 7C, 7D, 7E and 7F) with the barrel with the recess 11 is dislocated.

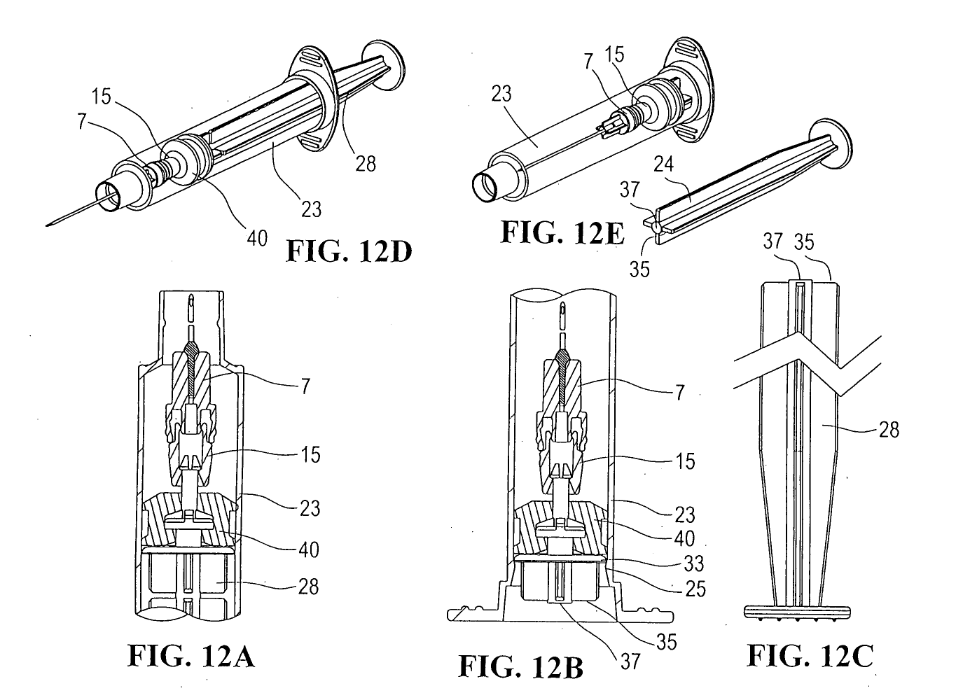

In Step 3 (shown in FIGS. 12A, 12B, 12C, 12D, 12E), as the hub cover 7 has dislocated fully with the barrel 23, the needle seat can be easily pulled to the barrel. When the thin circular seat 33 (FIGS. 8A, 8C) engages at the protruded rim 25, the plunger 28 cannot be pulled backward. At this instance, it will break at the breaking point 37 (FIGS. 8A, 8C), and the needle is contained within the barrel 23.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A retractable syringe comprising
(a) a cap having a cylindrical shape with a circular rim seat at one end and the external surface of the cap being formed with anti-slip elongated slots and the interior of the cap having a stepped circular hole and a semi-circular protruded rim;
(b) a hub cover having a stepped cylindrical post and a hole at the center thereof, a reinforcing rib provided to the stepped cylindrical post and an arch-shaped circular bottom seat being provided to the lower end of the stepped cylindrical post and at the half position of the circular bottom seat inwardly sloping being formed and the lower section of the bottom seat a recessed rim which is firmly engaged with the protruded rim being formed, and the lower section of the circular bottom seat having an arch-shaped protruded rim for firm engagement with the recessed rim at the hub bottom, and the inner circle of the circular bottom seat a guiding post being provided and the lower section of the guiding post being a protruded rim;
(c) a hub bottom extending the inwardly sloping bottom seat of the hub cover facilitating the engagement of the arch-shaped recessed rim and the protruded rim of the barrel and a cylindrical guiding holder being provided to the hub bottom which is mounted with the guiding post, a protruded rim being provided to the upper section of the inner circular of the guiding hole to control the distance of disengagement of the arch-shape recessed rim of the hub cover and the arch-shape protruded rim, and to fasten with the protruded rim of the hub cover facilitating the pulling of the hub cover back to the barrel, the inner section of the bottom seat having a conic hole and the upper section of the bottom seat being a recess which is in combination with the protruded rim of the hub cover and extends the inwardly sloping bottom seat of the hub cover, the conic hole at the bottom seat connected to a tapered circular hole;
(d) a barrel having a stepped cylindrical body having a hollow interior and the upper end of the inner opening provided with a protruded rim for engagement with the recessed rim of the hub cover and the lower section of the inner hole being a protruded rim for engagement with the circular rim seat of the plunger to avoid the needle seat from being pushed outside of the barrel and the oval shaped handle of the syringe being elongated protruded strap for preventing slipping;
(e) the plunger having a conic engaging hook and the center of the plunger being a V-shaped slot and the conic engaging hook for engagement with the inner engaging hook of the hub bottom so as to pull the hub cover and the hub bottom back to the barrel, the external of the circular thin rim seat being engagement with the protruded rim of the barrel to prevent needle seat being pushed out of the barrel, and the bottom of the thin circular seat being wing flap having a sectioned area for breakage, at the joining of one wing flap with another wing flap, a circular post being provided for breaking, and one end of the plunger having a circular handle with elongated protruded strap for prevention of slipping; and
(f) a gasket having a cylindrical shape with an interior having a center hole for connection with the circular rim seat of the plunger, and the circular protruded rim being used for water stopping.
